(19) United States
(12) Patent Application Publication
(10) Pub. No.: US 2021/0186422 A1
(43) Pub. Date: Jun. 24, 2021

(54) IMPLANTABLE MEDICAL DEVICE WITH METAL AND POLYMER HOUSING

(71) Applicant: Medtronic, Inc., Minneapolis, MN (US)

(72) Inventors: Christian S. Nielsen, River Falls, WI (US); Hailiang Zhao, Plymouth, MN (US); Kenneth D. Warnock, Manchester, MA (US)

(21) Appl. No.: 17/101,975

(22) Filed: Nov. 23, 2020

Related U.S. Application Data
(60) Provisional application No. 62/951,617, filed on Dec. 20, 2019.

(51) Int. Cl. A61B 5/00; H05K 5/04; H05K 5/06; A61B 5/042; A61B 5/0215

(52) U.S. Cl. CPC A61B 5/686; H05K 5/04; H05K 5/06; A61N 1/3956; A61B 5/0215; A61B 5/0022; A61B 5/042

(57) ABSTRACT

In some examples, manufacturing techniques for implantable medical devices are described. An example method may including positioning a metal housing component adjacent to a polymer housing component so that there is an interface between the metal housing component and the polymer housing component; and forming a seal at the interface between the metal housing component and the polymer housing component to join the metal housing component and the polymer housing component, wherein the joined metal housing component and the polymer housing component form at least a portion of housing for the implantable medical device, wherein the housing of the implantable medical device contains electronic circuitry.

This application claims the benefit of U.S. Provisional Patent Application No. 62/951,617, filed Dec. 20, 2019, the entire content of which is incorporated herein by reference.

### TECHNICAL FIELD

The disclosure generally relates to medical devices, and more particularly, to implantable medical devices.

### BACKGROUND

Various implantable medical devices (IMDs) have been clinically implanted or proposed for therapeutically treating or monitoring one or more physiological conditions of a patient. Such devices may be adapted to monitor or treat conditions or functions relating to heart, muscle, nerve, brain, stomach, endocrine organs or other organs and their related functions. Advances in design and manufacture of miniaturized electronic and sensing devices have enabled development of implantable medical devices capable of therapeutic as well as diagnostic functions such as pacemakers, cardioverters, defibrillators, biochemical sensors, and pressure sensors, among others. Such devices may be associated with leads to position electrodes or sensors at a desired location, or may be leadless, with the ability to wirelessly transmit data either to another device implanted in the patient or to another device located externally of the patient, or both.

In some examples, implantable miniature sensors have been proposed and used in blood vessels to measure directly the diastolic, systolic, and mean blood pressures, as well as body temperature and cardiac output. As one example, patients with chronic cardiovascular conditions, particularly patients suffering from chronic heart failure, may benefit from the use of implantable sensors adapted to monitor blood pressures. As another example, subcutaneously implantable monitors have been proposed and used to monitor heart rate and rhythm, as well as other physiological parameters, such as patient posture and activity level. Such direct in vivo measurement of physiological parameters may provide significant information to clinicians to facilitate diagnostic and therapeutic decisions. If linked electronically to another implanted therapeutic device (e.g., a pacemaker), the data may be used to facilitate control of that device. Such sensors also, or alternatively, may be wirelessly linked to an external receiver.

### SUMMARY

In some aspects, this disclosure describes implantable medical devices (IMDs) and example techniques for manufacturing such devices. The IMD may have a housing including a metal housing component joined to a polymer housing component along an interface between the two components. The housing of the IMD may contain electronic circuitry as well as other components such as a power source, e.g., that operates the medical device to sense one or more patient parameters or other operating functions of the 1 MB.

In some examples, the metal housing component and polymer housing component may be joined by positioning the respective components adjacent to each other along an interface, and then delivering energy to the metal housing component. Heat may be transferred to the polymer housing component from the metal housing component, e.g., via conductive heat transfer along the interface. The heating of the metal housing component may cause a portion of the polymer housing component to melt. The melted portion may wet on the surface of the metal housing component and then solidify by cooling to form the seal between the metal housing component and the polymer housing component. In some examples, the seal formed between the two components may be a hermetic seal.

In some examples, the disclosure is directed to a method for manufacturing the 1 MB. The method may include positioning a metal housing component adjacent to a polymer housing component so that there is an interface between the metal housing component and the polymer housing component. The method may further include forming a seal at the interface between the metal housing component and the polymer housing component to join the metal housing component and the polymer housing component, wherein the joined metal housing component and the polymer housing component form at least a portion of housing for the implantable medical device, wherein the housing of the implantable medical device contains electronic circuitry.

In some examples, the disclosure is directed to an 1 MB having electronic circuitry; and a housing, wherein the processing circuitry is contained within the housing, wherein the housing includes a metal housing component and a polymer housing component sealed to each other along an interface.

The details of one or more aspects of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A is a conceptual diagram illustrating an example medical device system, in accordance with some examples described in this disclosure.

FIG. 1B is a conceptual diagram illustrating another example medical device system, in accordance with some examples described in this disclosure.

FIG. 2 is a conceptual diagram illustrating an example IMD including metal and polymer housing components.

FIGS. 3A-3D are schematic diagrams illustrating an example IMD including metal and polymer housing components.

FIG. 4 is a flow diagram illustrating an example technique, in accordance with some examples described in this disclosure.

FIG. 5 is a schematic diagram illustrating a portion of an example IMD with an energy beam applied, in accordance with some examples described in this disclosure.

FIGS. 6-8 are micrographs showing cross-sectional views of various samples for experiments performed to evaluate aspects of the disclosure.

### DETAILED DESCRIPTION

As described above, the disclosure relates to implantable medical devices (IMDs) and example techniques for making IMDs. Various IMDs have been implanted or proposed for therapeutically treating or monitoring one or more physiological conditions of a patient. These IMDs may include a metal outer housing that contains electronic components capable of monitoring patient data, transmitting patient data, processing patient data, and/or delivering electrical stimulation into the body of the patient. The IMD may also include one or more electrodes located on the metal housing, e.g., to conduct electrical signals to and from the electronics within the metal housing. In some examples, the metal housing of the IMD may be formed of multiple metal housing components (e.g., top and bottom housing components) that are combined to form an outer housing of the IMD that forms a hermetically sealed enclosure for containing the electronics and other components of the IMD.

During the manufacturing process, the metal housing components may be joined to each other by a metal welding process. It may be important that metal housing components are adequately sealed to each other to protect the electronic components from liquid or vapor leaking into the device. Furthermore, having electrodes located on the outer metal housing may require complicated feedthroughs and/or other measures to electrically isolate the electrodes from the outer metal housing, e.g., to prevent shunting of electric fields sensed by the electrodes. However, the welding process to join two metal housing components as well as the design requirements to electrically isolate electrodes located on the metal housing may be relatively expensive.

In accordance with the disclosure, an IMD including a metal housing component and polymer housing component, and methods for manufacturing such IMD are described. The metal and polymer housing components may combine to form an enclosure within the outer housing of the IMD that contains electronic circuitry and/or other internal components of the 1 MB. In some examples, the IMD may include one or more electrodes on the polymer housing component. The polymer housing component may be formed of an electrically insulative polymer, e.g., to electrically isolate the electrodes from the metal housing component as well as electrically isolating respective electrodes from each other.

Examples of the disclosure may include techniques for joining a metal housing component to a polymer housing component to form an outer housing of the 1 MB. An example technique may include positioning a metal housing component adjacent to a polymer housing component, such that there is an interface between the two components. A seal may be formed at the interface between the metal housing component and polymer housing component by applying energy to the metal component to heat to the metal housing component. The heating of the metal housing component may in turn heat the polymer housing component to an elevated temperature at the interface, e.g., where the elevated temperature is equal to or greater than a melting temperature (and, e.g., lower than the decomposition onset temperature) of the polymer housing component. The elevated temperature may melt the polymer housing component to reflow in the area of the interface and wet the surface of the metal housing component at the interface. Upon cooling of the polymer, a seal may be formed between the metal housing component and the polymer housing component.

In some examples, the method of heating the metal housing component may include a laser welding process or other process in which a laser or other energy source is directed at the surface of the metal housing component. In one example, a laser welding process may be used in which a pulsed laser beam or continuous wave laser beam may be directed to a surface of the metal housing component to heat the metal housing component and, as a result, melt or otherwise cause a portion of the polymer housing component to reflow at an interface between the polymer housing component and metal housing component. In one example, the laser beam or other energy source may move relative to the metal housing component while forming the hermetic seal. As will be described below, other suitable energy sources and/or heating techniques may be employed in such a process and may include, e.g., inductive energy sources or process which furnace heating is employed to heat the metal part with subsequent insertion/assembly with the polymer housing component, resistance heating by current applied to the metal housing component, friction against the surface of the metal housing component, RF heating, heat from another focused light, hot air, conductive heat transfer or other heat transfer from contact, ultrasonic energy, and/or the like.

In some examples, the IMD having the combined metal and polymer housing components may include a miniaturized implantable medical device configured to sense various physiological parameters of a patient, such as one or more physiological pressures, electrical signals, and the like. Such devices may include a hermetic housing that contains a power source and electronic circuitry to operate the 1 MB. In some examples, the IMD may include one or more electrodes each defined by an electrically conductive surface on an outer surface of the polymer housing component. In some examples, the IMDs may include processing circuitry configured to at least sense electrical signals via the electrode(s). In some examples, the IMD may include processing circuitry configured to at least control delivery of electrical stimulation via the electrode(s).

In some examples, an IMD including metal and polymer housing components joined by a seal to define the outer housing of the IMD may provide one or more advantages. As noted above, the polymer housing component may function to electrically isolate the one or more electrodes on the housing from the metal housing component and/or other electrodes of the 1 MB. Additionally, or alternatively, a controlled process to join the polymer and metal housing components using an energy source to heat the metal component to melt the polymer housing at an interface between the components may allow for improved manufacturability and improved manufacturing efficiency. Joining a metal housing component and polymer housing component to form a hermetically sealed IMD housing may be performed with more precision and replicability than other sealing methods that involve an additional material such as a polymer adhesive. An IMD containing a hermetically sealed metal housing component and polymer housing component may prevent any disruptions in IMD performance for the entirety of the operating life of the device. An IMD containing a hermetically sealed metal housing component and polymer housing component may reduce the number of foreign materials introduced into a patient's body, and ensure the proper function of the IMD.

For ease of description, examples of the present disclosure are primarily described with regard to miniaturized sensing medical devices configured to be implanted within the heart of a patient, e.g., to monitor a pressure within the heart of the patient. However, examples are not limited to such devices and configurations. Other medical devices including multi-component outer housings, e.g., that house internal components within a hermetically sealed enclosure are contemplated. In some examples, the multi-component housings may be employed with IMDs such as implantable cardiac devices that deliver pacing, defibrillation and/or cardioversion therapy, or implantable medical devices that delivery neurostimulation therapy to patient. In some examples, the IMD may be configured to deliver electrical stimulation therapy to a patient, e.g., via one or more electrically coupled leads, and/or may sense bioelectrical signals of the patient. In other examples, the IMD may sense one or more physiological parameters of a patient but not delivery electrical therapy to the patient, e.g., to monitor one or more cardiac parameters of a patient without delivering electrical therapy to the patient. Other functions of an IMD of this disclosure beyond electrical sensing/stimulation may include capturing chemical parameters (e.g., glucose or oxygen sensor), capturing images, providing navigation assistance when delivering the device, and/or delivering fluids/other bioactive items.

In some examples, the medical device may be an IMD that is targeted for a relatively short-term implant in a patient and/or IMD that are relatively easy to explant from a patient. In some examples, the IMD may function the same or substantially similar to Reveal LINQ™ ICM, available from Medtronic plc, of Dublin, Ireland. In some examples, medical devices of this disclosure may be configured to be implanted in a patient subcutaneously, subpectorally, and/or in any other suitable implant location.

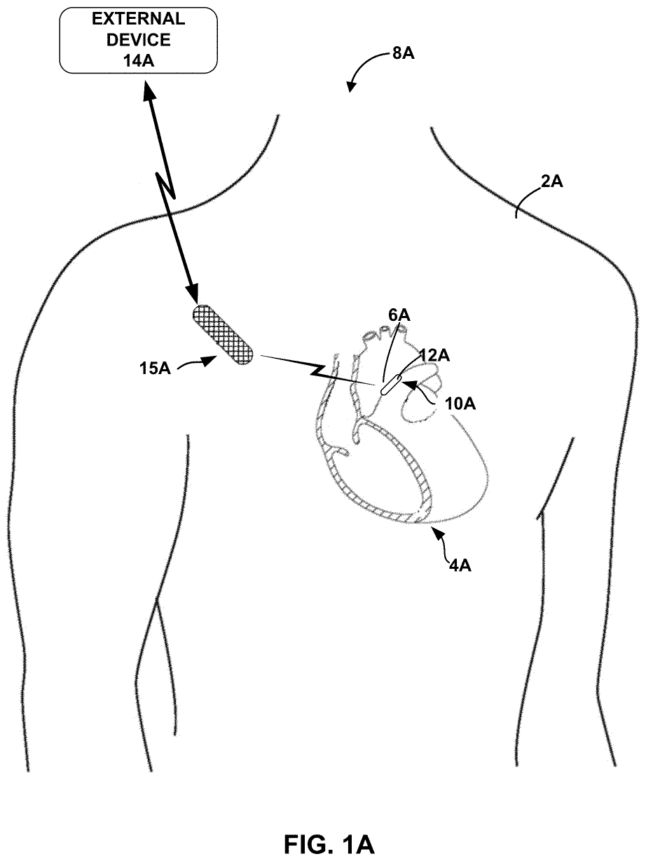

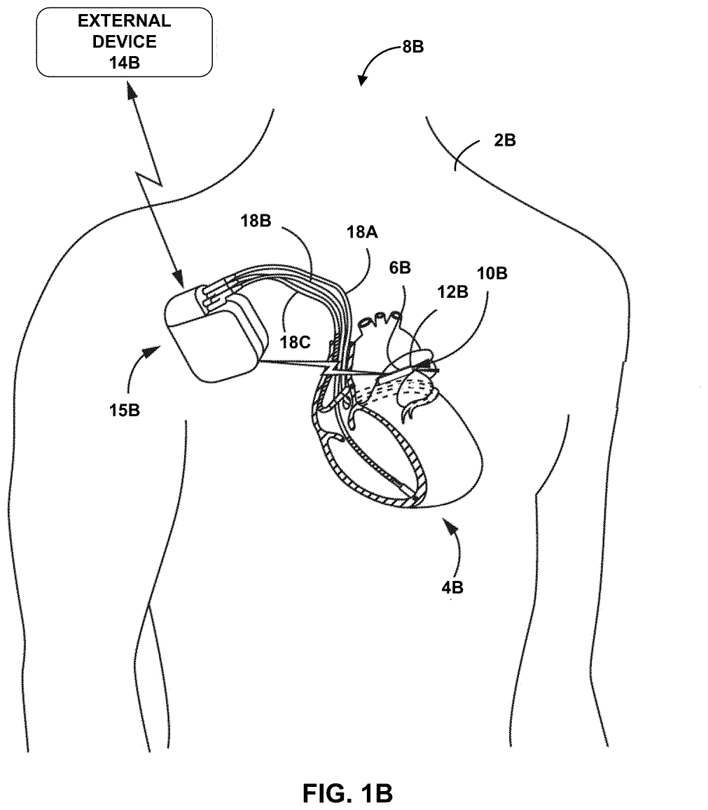

FIG. 1A is a conceptual diagram illustrating an example of a medical device system 8A. Medical device system 8A includes IMD 15A, which is implanted within patient 2A, and external device 14A. IMD 15A may comprise an implantable or insertable cardiac monitor or an implantable hub device, in communication with external device 14A. Medical device system 8A also includes implantable sensor assembly 10A, which comprises pressure sensing device 12A. As shown in FIG. 1A, implantable sensor assembly 10A may be implanted within pulmonary artery 6A of heart 4A of patient 2A. In some examples, pulmonary artery 6A of heart 4A may comprise a left pulmonary artery, whereas in other examples, pulmonary artery 6A may comprise a right pulmonary artery. For the sake of clarity, a fixation assembly for sensor assembly 10A is not depicted in FIG. 1A. IMD of this disclosure are not limited to those configured to be implanted in a heart of a patient.

IMD 15A comprises an insertable cardiac monitor (ICM) configured to sense and record cardiac electrogram (EGM) signals from a position outside of heart 4A, and will be referred to as ICM 15A hereafter. In some examples, ICM 15A includes or is coupled to one or more additional sensors, such as accelerometers, that generate one or more signals that vary based on patient motion, posture, blood flow, or respiration. ICM 15A may monitor a physiological parameter such as posture, heart rate, activity level, or respiration rate, and may do so at times when the one or more additional sensors, such as pressure sensing device 12A, is configured with circuitry to measure the cardiovascular pressure of patient 2A. ICM 15A may be implanted outside of the thoracic cavity of patient 2A, e.g., subcutaneously or submuscularly, such as at the pectoral location illustrated in FIG. 1A. In some examples, ICM 15A may take the form of a Reveal LINQTAIICM, available from Medtronic plc, of Dublin, Ireland.

ICM 15A may transmit posture data, and other physiological parameter data acquired by ICM 15A, to external device 14A. ICM 15A also may transmit cardiovascular pressure measurements received from pressure sensing device 12A to external device 14A. External device 14A may be a computing device configured for use in settings such as a home, clinic, or hospital, and may further be configured to communicate with ICM 15A via wireless telemetry. For example, external device 14A may be coupled to a remote patient monitoring system, such as Carelink®, available from Medtronic plc, of Dublin, Ireland. External device 14A may, in some examples, comprise a programmer, an external monitor, or a consumer device such as a smart phone.

External device 14A may be used to program commands or operating parameters into ICM 15A for controlling its functioning, e.g., when configured as a programmer for ICM 15A. External device 14A may be used to interrogate ICM 15A to retrieve data, including device operational data as well as physiological data accumulated in the memory of ICM 15A. The accumulated physiological data may include cardiovascular pressure generally, such as one or more of a systolic pressure, a diastolic pressure, and a mean pulmonary artery pressure, or medians of such pressures, although other forms of physiological data may be accumulated. In some examples, the interrogation may be automatic, e.g., according to a schedule. In other examples, the interrogation may occur in response to a remote or local user command. Programmers, external monitors, and consumer devices are examples of external devices 14A that may be used to interrogate ICM 15A.

Examples of wireless communication techniques used by ICM 15A and external device 14A include radiofrequency (RF) telemetry, which may be an RF link established via an antenna according to Bluetooth®, Wi-Fi™, or medical implant communication service (MICS), or transconductance communication (TCC), which may occur via electrodes of ICM 15A. Examples of wireless communication techniques used by ICM 15A and pressure sensing device 12A may also include RF telemetry or TCC. In one example, ICM 15A and pressure sensing device 12A communicate via TCC, and ICM 15A and external device 14A communicate via RF telemetry.

Medical device system 8A is an example of a medical device system configured to monitor a cardiovascular pressure of patient 2A. Although not illustrated in the example of FIG. 1A, a medical device system may include one or more implanted or external medical devices in addition to or instead of ICM 15A and pressure sensing device 12A. For example, a medical device system may include a vascular implantable cardiac defibrillator (ICD) or pacemaker (e.g., IMD 15B illustrated in FIG. 1B). One or more such devices may generate physiological signals, and may include processing circuitry configured to monitor cardiovascular pressure. In some examples, the implanted devices may communicate with each other or with external device 14A.

FIG. 1B is a conceptual diagram illustrating another example medical device system 8B. Medical device system 8B includes sensor assembly 10B implanted, for example, in left pulmonary artery 6B of patient 2B, through which blood flows from heart 4B to the lungs, and another device, such as a pacemaker, defibrillator, or the like, referred to as IMD 15B. For purposes of this description, knowledge of cardiovascular anatomy is presumed, and details are omitted except to the extent necessary or desirable to explain the context of the disclosure.

In some examples, IMD 15B may include one or more leads 18A-18C, that carry electrodes that are placed in electrical contact with selected portions of the cardiac anatomy in order to perform the functions of IMD 15B, as is well known to those skilled in the art. For example, IMD 15B may be configured to sense and record cardiac EGM signals via the electrodes on leads. IMD 15B may also be configured to deliver therapeutic signals, such as pacing pulses, cardioversion shocks, or defibrillation shocks, to heart 4B via the electrodes. In the illustrated example, IMD 15B may be a pacemaker, cardioverter, or defibrillator.

In some examples, this disclosure may refer to IMD 15B, particularly with respect to its functionality as part of a medical device system that monitors cardiovascular pressure and other physiological parameters of a patient 2B, as an implantable monitoring device or implantable hub device. In some examples, IMD 15B includes or is coupled to one or more additional sensors, such as accelerometers, that generate one or more signals that vary based on patient motion or posture, blood flow, or respiration. IMD 15B may monitor posture of patient 2B at or near the times when implantable pressure sensing device 12B is measuring cardiovascular pressure.

IMD 15B also may have wireless capability to receive and transmit signals relating to the operation of the device. IMD 15B may communicate wirelessly to an external device, such as external device 14B, or to another implanted device such as pressure sensing device 12B of the sensor assembly 10B, e.g., as described above with respect to IMD 15A, external device 14A, and pressure sensing device 12A of FIG. 1A. In some examples, the pressure sensing device may communicate wirelessly and directly with external device 14B, rather than communicating with external device 14B through the IMD 15B.

Medical device system 8B is an example of a medical device system configured to monitor the cardiovascular pressure of patient 2B. One or more of IMD 15B, implantable pressure sensing device 12B, and external device 14B, individually, or collectively, may include processing circuitry that allows medical device system 8B to function as described herein. Such function may include measuring a cardiovascular pressure of patient 2B, such as pulmonary artery pressure. In some examples, an implantable pressure sensing device 12 measures the cardiovascular pressure at a plurality of predetermined times during a day or a portion of a day, e.g., at night.

Sensor assembly 10A and sensory assembly 10B may include outer housings (not labelled in FIGS. 1A and 1B) that contain one or more components such as electronic circuitry and a power source. In some examples, the electronic circuitry may include, e.g., processing circuitry, telemetry circuitry, and/or the like, which allow assemblies 10A and 10B to operate as described herein. As will be described further below, the outer housings may be formed of at least one metal housing component and at least one polymer housing component. A seal may be formed between a metal housing component and a polymer housing component along an interface between the two components. In some examples, the seal may be a hermetical seal between the two components, e.g., to allow for the internal components to be contained within a hermetically sealed housing enclosure. In some examples, the seal between the components may be formed by positioning the components adjacent to each other along an interface, and then delivering energy to the metal housing component. Heat may be transferred to the polymer housing component from the metal housing component, e.g., via conductive heat transfer along the interface. The heating of the polymer housing component may cause a portion of the polymer housing component to melt. The melted portion may wet on the surface of the metal housing component and then solidify by cooling to form the seal between the metal housing component and the polymer housing component.

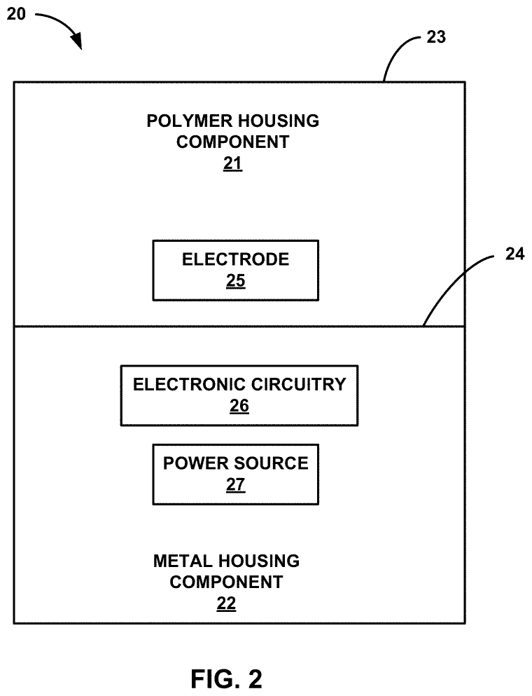

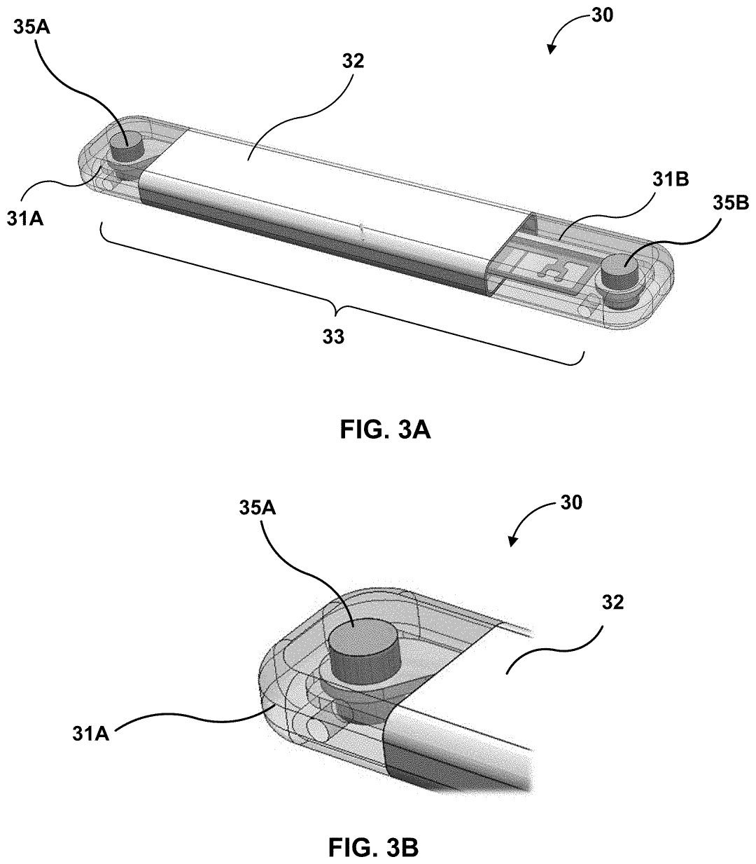

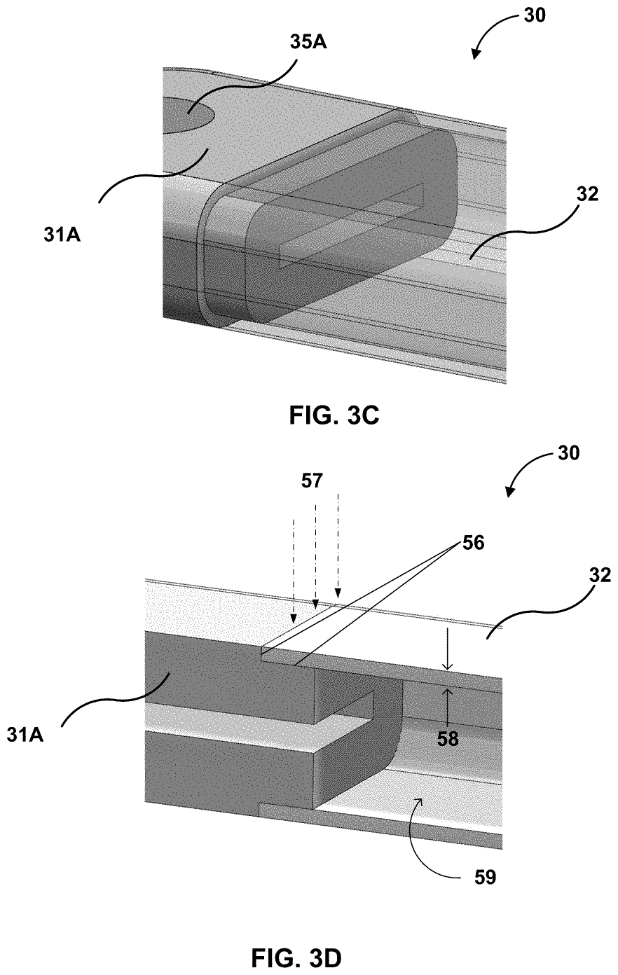

FIG. 2 is a conceptual diagram illustrating example IMD 20 including outer housing 23. Housing 23 is defined by a combination of metal housing component 22 and polymer housing component 21, in accordance with some examples described in this disclosure. Sensor assemblies 10A and 10B are examples of IMD 20. Housing 23 defines an enclosure that houses internal components such as electronic circuitry 26 and power source 27. In the example of FIG. 3, electronic circuitry 26 and power source 27 are shown being within metal housing component 22. However, electronic circuitry 26 and power source 27 (as well as other internal components) may be located within either metal housing component 22, polymer housing component 21 or a combination of the components (e.g., as metal housing component 22 and polymer housing component 21 may combine to define an overall hermetically sealed enclosure that contains the internal components).

Electrode 25 may define an electrically conductive surface that forms a portion of the outer surface of polymer housing component 21. In cases in which polymer housing component 21 is formed of an electrically insulating material, polymer housing component 21 may function to electrically isolate electrode 25 from metal housing component 22. While a single electrode is shown in FIG. 2, IMD 20 may include multiple electrodes electrically isolated from each other and from metal housing component 22. In some examples, electrode 25 may be formed of a biocompatible conductive material. For example, electrode 25 may be formed from any of stainless steel, titanium, platinum, iridium, or alloys thereof. In addition, electrode 25 may be coated with a material such as titanium nitride or fractal titanium nitride, although other suitable materials and coatings for electrodes may be used.

Electrode 25 may be electrically coupled to electronic circuitry 26 and/or power source 27 within housing 23. Using electrode 25, electronic circuitry 26 and/or power source 27, IMD 20 may sense electrical signals of a patient in which IMD 20 is implanted and/or deliver electrical signals to the patient.

Although not shown, IMD 20 may include telemetry circuitry to allow IMD 20 to communicate with other devices located internal or external to the patient. Under the control of the electronic circuitry 26 of IMD 20, the telemetry circuitry may receive downlink telemetry from and send uplink telemetry to external devices with the aid of an antenna, which may be internal and/or external.

In some examples, IMD 20 may also include one or more other sensors configured to sense one or more physiological parameters of the patient.

Electronic circuitry 26 may include or may be one or more processors or processing circuitry, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” and “processing circuitry” as used herein may refer to any of the foregoing structure or any other structure suitable for implementation of the techniques described herein.

Depending on the function of IMD 20, electronic circuitry 26 may include signal sensing circuitry and/or electrical signal generating circuitry.

Although not shown, IMD 20 may include a memory within housing 23. The memory may include any volatile or non-volatile media, such as a random-access memory (RAM), read only memory (ROM), non-volatile RAM (NVRAM), electrically erasable programmable ROM (EEPROM), flash memory, and the like. The memory may be a storage device or other non-transitory medium. In general, memory of IMD 20 may include computer-readable instructions that, when executed by a processing circuitry of IMD 20, cause it to perform various functions attributed to the device herein. For example, processing circuitry of IMD 30 may control the signal generator and sensing circuitry according to instructions and/or data stored on memory to deliver therapy to a patient and perform other functions related to treating condition(s) of the patient with IMD 20.

The various components of IMD 20 may be coupled to a power source 27. Power source 27 may be any suitable power source that provides operational power to IMD 20. In some examples, power source 27 may be a primary or secondary battery, such as a lithium battery. Power source 27 may be capable of holding a charge for several years.

While a single polymer housing component 21 and a single metal housing component 22 are shown for IMD 20, examples are contemplated in which housing 23 includes more than one metal housing component 22 and/or more than one polymer housing component 21 joined to each other. Furthermore, while a single electrode 25 is shown in FIG. 2, in some examples, IMD 20 may include multiple electrodes 25 on the same or different polymer housing components 21 of IMD 20.

In some examples, polymer housing component 21 and metal housing component 22 may be joined together by one or more of the example methods disclosed herein, e.g., such that a hermetic seal is formed between the two components, thus protecting the internal components from fluids such as body fluids and/or gases. In the example depicted in FIG. 2, electrical feedthroughs (not shown) may provide electrical connection of electrode 25 to circuitry within housing 23. Polymer housing component 21 and metal housing component 22 may be joined together at interface 24. The seal formed at interface 24 between the respective housing components may define a hermetic seal that hermetically seals component within housing 23 from the environment external to housing 23.

Polymer housing component 21 may be formed of a polymeric material. Any suitable polymer or combination of polymers may be used for polymer housing component 21. The polymeric material may be a biocompatible polymer suitable for implantation in a patient. The polymeric material may be a material that forms a hermetic boundary between the environment external to housing 23 and the internal components. In some examples, the polymer material may have a relatively low permeability (e.g., to form a hermetic barrier). As described herein, polymer housing component 21 may be formed of a polymeric material that melts when heated, e.g., by heat transferred to polymer housing component 21 from metal housing component 22 along interface 24. In some examples, the polymer housing component 21 may be formed of a polymer that is able to reflow and solidify without significant degradation. In some examples, the polymer may be a thermoplastic.

In some examples, polymer housing component 21 includes a single polymer material. In other examples, polymer housing component 21 includes a combination of polymers. Suitable polymers may include polyether ether ketone, polysulfone, polyetherimide, polyphenylsulfone, ultra-high molecular weight (UHMW) polyethylene (PE), and/or polyethersulfone (PES). Other suitable polymers may include those which are liquid crystalline polymers (LCPs), which may be highly adaptable to IMD applications. In some examples, polymeric housing component includes at least one polymeric polymer. In some examples, polyolefins and/or silicones may be employed for polymer housing component 21.

In some examples, polymer housing component 21 may be formed of bulk or main polymer portion (e.g., PEEK or LCP) with a layer of a second polymer material (e.g., a suitable thermoplastic) that has a lower melting temperature in the area of contact with metal housing component 22 (e.g., at interface 24). In some examples, the second polymer material may be referred to a “tie layer,” and when melted and cooled, may have better adhesive properties than the bulk material to ensure a better bond with metal housing component 22.

In some examples, a suitable polymer material may be selected based on how the material expands when heated. For example, a polymer with a chemical foaming agent blended in just above the melt temperature, but then begins to foam at a higher temperature may be selected. This foaming action may both increase the internal pressure inside the device (e.g., helping to force the polymer out), as well as helping ensure a better bond to the inside.

Metal housing component 22 may be formed of any suitable metal or alloy or combination of metals or alloys. Like that of polymer housing component 21, metal housing component 22 may be formed of one or more metals and/or alloys that is biocompatible for implantation into a patient. The metal or alloy material may be a material that forms a hermetic boundary between the environment external to housing 23 and the internal components. In some example, the metal or alloy material may have surface morphology that has a low reflection for the outer surface of housing component 22. For the surface of metal housing component 22 it may be desirable for a low surface roughness that wets well, e.g., to increase contact with reflow material from polymer housing component 21. Suitable metal or alloy materials may include at least one of stainless steel, titanium (e.g., grades 1, 5, 9, 23, and the like), tantalum, niobium, platinum, or iridium. In some examples, a metal or alloy may be selected that has desirable thermal behavior (e.g., in terms of conduction/absorption from lasers in a laser heating process).

In some examples, metal housing component 22 may have surface modifications or other properties, e.g., surface roughness, cleanliness, oxides, in the area of interface 24 with polymer housing 21 that promote better bonding with the polymer. In some examples, larger scale features, like dovetail grooves, ridges, partial or even through holes, may serve to help seal/lock the polymer to the metal housing after being joined as described herein. Locking and/or sealing between the respective components may also be improved by mechanisms that tend to force the polymer housing component 21 into close contact with the metal housing component 22 once the polymer melts. Foaming agents in the polymer as described above may be one example mechanism, but other mechanisms may be employed. For example, a preloaded compression spring may be placed inside polymer housing component 21 in the area of interface 24, e.g., at the rectangular hole in the center of polymer housing 31A shown in FIG. 3C. Once the polymer housing 31A is softened by an external laser beam heating of metal housing 32, the spring may deform the softened polymer into closer contact with the metal housing. Other approaches include making the polymer and metal housings components a relatively tight fit, and assembling the respective components inside a chamber with higher than atmospheric pressure so that pressure is captured inside. With the appropriate design, reheating the polymer outside the pressure chamber might cause the polymer to melt and be forced into good contact with the metal housing as a technique to improve the bond in the area of interface 24.

FIGS. 3A-3D are conceptual schematic diagrams illustrating various view of example IMD 30 including polymeric housing components 31A and 31B, and metal housing component 32. IMD 30 may be an example of IMD 20 of FIG. 2. In some examples, IMD 30 may be configured to function as a monitoring device, such as ICM 15A, pressure sensing device 12A, or pressure sensing device 12B, or as a device that monitors and/or delivers electrical therapy to a patient, such as IMD 15B described above. In FIGS. 3A and 3B, polymer housing components 31A and 31B are shown as being semitransparent for illustrative purposes, e.g., to show the one or more internal components of IMD 30. FIG. 3C metal housing 32 is shown as being semitransparent and without internal components for illustrative purposes. FIG. 3D is a cross-sectional view of portion of IMD 30 along the longitude axis of IMD 30. FIG. 3D does not show the internal components of IMD 30 but instead only show polymer housing component 31A and metal housing component 32.

IMD 30 includes outer housing 33 which may be the same or substantially similar to that described above for housing 23 of IMD 20 in FIG. 2. For examples, housing 33 includes first and second polymeric housing components 31A and 31B, which may be the same or substantially similar to that described for polymeric housing component 21 in FIG. 2. First polymer housing component 31A and second polymer housing component 31B may have substantially the same composition (e.g., formed of the same polymer composition) or may have different compositions (e.g., formed from different polymer compositions).

Housing 33 also includes metal housing component 32, which may be the same or substantially similar to that described for metal housing component 22 in FIG. 2. Metal housing component 32 may have a tubular shape that define internal cavity 59 to house all or a portion of one or more of the internal components of IMD 30. First polymer housing component 31A is joined at one open end of metal housing component 32 and closes off that open end of metal housing component 32. For example, as shown in FIG. 3D, first polymer housing component 31A is joined to metal housing component 32 along interface 56. During assembly of IMD 30, a seal such as a substantially hermetic seal may be formed between first polymer housing component 31A and metal housing component 32 at interface 56 when first polymer housing component 31A and metal housing component 32 are joined to each other.

Likewise, second polymer housing component 31B is joined at the other open end of metal housing component 32 and closes off that open end of metal housing component 32. Thus, in combination, housing components 32, 31A and 31B may form an outer housing 33 for IMD 30 that defines a sealed enclosure, e.g., a hermetically sealed enclosure, having inner cavity 59 that houses one or more components of IMD 30. For example, like that described above for IMD 20, housing 33 of IMD 20 may contain electronics and other internal components necessary or desirable for executing the functions associated with the device. In one example, housing 33 of IMD 30 includes one or more of processing circuitry, memory, a signal generation circuitry, sensing circuitry, telemetry circuitry, and a power source. In some examples, housing 33 encloses electronic circuitry 26 and protects the circuitry contained therein from fluids such as body fluids.

IMD 30 also includes two electrodes (first electrode 35A and second electrode 35B), which may the same or substantially similar to that described for electrodes 25 of IMD 20. First and second electrodes 35A and 35B may be used by IMD 30 to sense electrical signals within a patient and/or delivery electrical signals generated by IMD 30 to one or more target sites within a patient. For example, first and second electrodes 35A and 35B may be used to sense cardiac EGM signals, e.g., ECG signals, when IMD 30 is implanted in the patient either sub-muscularly or subcutaneously. The signals may be sensed by IMD 30 using a unipolar or multipolar configuration. In some examples, the EGM signals may be stored in a memory of the IMD 30, and data derived from the cardiac EGM signals may be transmitted via an integrated antenna to another medical device, which may be another implantable device or an external device, such as external device 14A. In some examples, IMD 30 may function the same or substantially similar to that of Reveal LINQ® Insertable Cardiac Monitor (available from Medtronic plc., Dublin, IE).

As shown, first electrode 35A is positioned on first polymer housing component 31A and second electrode 35B is positioned on second polymer housing component 31B. As described above, first polymer housing component 31A and second polymer housing component 31A may each be formed of an electrically insulating material. In this manner, first polymer housing component 31A may electrically isolate first electrode 35A from metal housing component 32 and second electrode 35B. Similarly, second polymer housing component 31A may electrically isolate second electrode 35B from metal housing component 32 and first electrode 35A. While the examples of first and second electrodes 35A and 35B are shown as being located on the same major surface of housing 33 at distal and proximal ends of IMD 30, respectively, and as defining flattened, outward facing conductive surfaces, other examples are contemplated. For example, one or both of first and seconds electrodes 35A and 35B may extend from first major surface, around rounded edges or an end surface, and onto the second major surface. Thus, the electrode may have a three-dimensional curved configuration. In some examples, all or a portion of first electrode 35A may be located on first major surface of housing 33 and all or a portion of second electrode 35B may be located on a second major surface of housing 33.

During the manufacturing process for IMD 30, first electrode 35A and first polymer housing component 31A may be formed separately from metal housing component 32. The composite assembly of first electrode 35A and first polymer housing component 31A may then be joined to metal housing component 32. For example, the electrically conductive structure of first electrode 35A (and associated feedthroughs and other structure) may be fabricated and then the polymer material of first polymer component 31A may be backfilled and/over-molded around the prefabricated structure. The composite component of first electrode 35A and first polymer housing component 31A may then be joined to metal housing component 32 along interface 56. The composite structure of second electrode 35B and second polymer housing component 31B may be similarly manufactured, and subsequently joined to metal housing component 32 at the opposite end of housing component 32. First polymer housing component 31A and second polymer housing component 31B may each be joined to metal housing component 32 to form outer housing 33 of IMD 30, e.g., using one or more of the example techniques described herein.

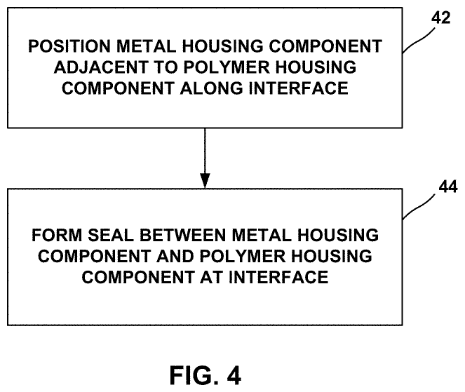

FIG. 4 is a flow diagram illustrating an example technique for assembling an IMD, in accordance with some examples described in this disclosure. The example technique shown in FIG. 4 may be used to form an IMD having an outer housing made from one or more polymeric housing components and one or more metal housing components that are sealed to each. The example technique shown in FIG. 4 may be used to assemble the respective housing components of IMD 20 or IMD 30 described above. For ease of description, the example technique of FIG. 4 is described with regard to the joining of first polymer housing component 31A to metal housing component 32 for IMD 30. However, it is recognized that such a process may be used to join second polymer housing component 31B to metal housing component 32 at the opposite end of metal housing 32 and/or may be used to assemble any housing that includes a polymer housing component and a metal housing component joined to each other, e.g., to form a substantially hermetic seal.

As shown in FIG. 4, the example technique includes positioning metal housing component 32 adjacent to first polymer housing component 31A, e.g., so that the respective components are directly adjacent to each other along interface 56 (42). Such an arrangement is shown, e.g., in FIGS. 3C and 3D. In some examples, positioning metal housing component 32 adjacent to first polymer housing component 31A (42) may include contacting surfaces of the polymeric and metal housing components 31A and 32 with each other at interface 56.

Any suitable technique may be employed to position metal housing component 32 and first polymer housing component 31A as described. For example, the respective components may be manually positioned adjacent to each other or automated robotic equipment may be employed to position the respective components as described. In some examples, metal housing component 32 and first polymer housing component 31A may be sized, shaped, and/or otherwise configured such that there is press fit (also referred to as an interference fit) formed at interface 56 to secure (e.g., temporarily hold) metal housing component 32 and first polymer housing component 31A to each other (e.g., so a seal may be formed between the two components as describe below).

Once metal housing component 32 has been positioned adjacent to first polymer housing component 31A (42), a seal may be formed between metal housing component 32 and first polymer housing component 31A at interface 56 (44). For example, energy (represent by arrows 57 in FIG. 3D) may be applied to metal housing component 32, e.g., in an area at or near interface 56, such that the temperature of metal housing component 52 increases. As a result, energy, e.g., in the form of heat, may then be transferred from metal housing component 32 to first polymer housing component 31A in the area of interface 56 (e.g., via conductive and/or convective heat transfer). The transferred energy may increase the temperature of first polymeric housing component 31A at or near interface 56 to a threshold temperature at which the polymeric material of first polymer housing component 31A softens and/or melts. Once softened and/or melted, the polymer material may reflow along interface 56 so that a seal if formed by between first polymer housing component 31A and metal housing component 32 when the polymer material cools (e.g., by terminating the application of the energy source applied to metal housing 32). In some examples, the temperature of first polymeric housing 31A in the area of interface 56 may be increase to or above the glass transition temperature of the polymer material and/or increase to or above the melting temperature and/or softening temperature of the polymer material. In some examples, the reflow of the polymeric material increases the contact between the adjacent surfaces of first polymer housing component 31A (e.g., as compared to the contact between the surfaces prior to application of the energy to metal housing component 32). In some examples, contact between the surfaces increases as a result of this “wetting” of the surface of metal housing component 32 in contact with softened and/or melted polymer material along interface 56.

In some examples, the seal formed along interface 56 by the process of FIG. 4 may be a substantially hermetic seal. In some examples, a helium leak test may be employed to evaluate the hermeticity. A substantially hermetic seal may be defined by such a test with helium transfer below detectable levels at time=0. However, hermeticity of a seal may be measured using other metrics, e.g., pressure decay/pressure increase tests by creating a pressure differential between inside and outside of the housing.

Any suitable energy source may be employed to apply energy 57 to metal housing 32 (44). For example, a laser beam source may be employed that applies laser beam energy to the metal housing component 32, in accordance with this disclosure. In some examples, the process may be a laser beam welding process. A laser energy source may offer desirable control and targeting for applied energy 57. Other forms of heat sources and/or heating techniques may be employed and may include electron beam, electrical arc, plasma, resistance heating (e.g., by current applied to the metal housing component), electrical heating tools, inductive heating, pre-heating (e.g., in a furnace), friction against the surface of the metal housing component, RF heating, heat from another focused light, hot air, conductive heat transfer or other heat transfer from contact, ultrasonic energy, and/or the like.

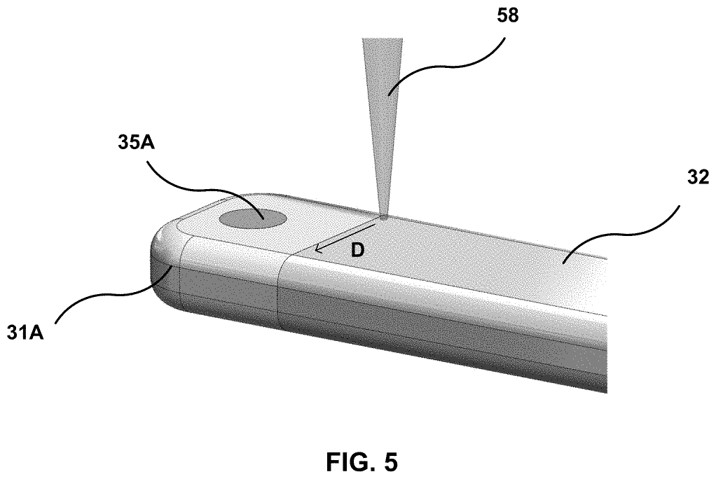

FIG. 5 is a conceptual schematic diagram illustrating that application of laser beam energy 58 (or other type of suitable energy from an external source) to metal housing component 32 in the area of interface 56 (shown in FIG. 3D). To apply beam energy 58 to metal housing component 32 (44), beam energy 58 may be moved relative to metal housing along direction D in a substantially continuous or periodical fashion over the entire outer perimeter of metal housing 32 in the area of interface 56. For example, energy 58 may be stationery and metal housing component 32 and first polymer housing component 31A may be moved, or vice versa. Additionally, or alternatively, energy 58 may be moved and metal housing component 32 and first polymer housing component 31A may be stationary. By moving the energy 58 along direction D all portions of metal housing component 32 may be heated and the heating may be controlled as desired.

During the process, energy 58 may be applied on a substantially continuous or periodic basis. In some examples, energy 58 may be applied according to an on/off duty cycle. The timing of the application of energy 58 and/or the relative movement of energy 58 relative metal housing component 32 (as well as other parameters such as beam energy source diameter, power, and the like) may be selected such that enough energy is delivered to metal housing component 31 to heat the adjacent polymer material of first polymer housing component 31A to a temperature sufficient to form a seal (e.g., a substantially hermetic seal) along interface 56 upon cooling of the polymer material.

In some examples, sufficient heat is transferred to first polymeric housing component 31A from metal housing component 32 to increase the temperature of the polymeric housing component to or above the glass transition temperature (Tg) of the polymer material. In some examples, sufficient heat is transferred to first polymeric housing component 31A from metal housing component 32 to increase the temperature of the polymeric housing component to or above the melting temperature of the polymer material. In some examples, sufficient heat is transferred to first polymeric housing component 31A from metal housing component 32 to increase the temperature of the polymeric housing component to or above the softening temperature of the polymer material. When the polymer that forms polymeric housing component 31A reaches a temperature at or above its Tg, melting, and/or softening temperature, the polymer reflows and forms a seal with metal housing component 32 along interface 56 upon cooling.

Energy 58 may be applied using any suitable parameters for performing the process described for FIG. 4. The parameters for energy 58 may be dependent on a number of factors including, e.g., the composition (and other heat transfer properties such as thickness 58) of metal housing component 32 and the composition of polymer housing component 31A. In some examples, when energy 58 is in the form of a continuous wave laser beam energy, energy 58 may have a power of about 10 Watts (W) to about 1000 W, such as about 100 W to about 300 W; a beam diameter of about 0.001 inches to about 0.030 inches, such as about 0.008 inches to about 0.026 inches. In some examples, energy source 58 may move relative to metal housing component 32 as a rate of about 1.0 inch per minute (ipm) to about 500 ipm, such as about 50 ipm to about 150 ipm. Other values are contemplated. Energy 58 can also be in the form of pulsed laser beam energy.

The application of energy 58 may be controlled to increase the temperature of the material of polymer housing component 31A above the softening and/or melting point of the material but below a threshold maximum temperature for metal housing component 32 and/or polymer housing component 31A. The threshold maximum temperature may be a temperature at which metal housing component 32 and/or polymer housing component 31A that cause undesirable side effects to the housing components. For example, first electrode 35A may include a thermally sensitive component. In such cases, it may be desirable to design the polymeric housing component using a polymer having a Tg, melting point, and/or softening point that is lower than a temperature which would harm first electrode 35A (or other thermally sensitive component of IMD 30). For example, it may be known that temperatures above 150° C., need to be avoided when using a given thermally sensitive component. In such cases, the polymeric housing component may include a polymer having a Tg, melting point, and/or softening point of less than about 150° C.

In some examples, the temperature of the polymer housing component 31A may be kept below the onset of polymer degradation or decomposition. The bonding strength may decrease when the polymer decomposes and may ultimately lead to loss of hermeticity.

In some examples, the temperature during the process may be controlled to keep the temperature below the degradation temperature of other polymer components inside the housing (e.g., of other internal polymer seals between battery cathode/anode), below a distortion temperature of polymer housing component 31A, and/or degradation temperature of circuit devices of IMD 31A.

In some examples, employing a laser as the energy source may be beneficial as it may provide control over the intensity of the energy, the duration that it is applied over, and/or the ability to focus the application of the energy to very specific areas so other areas remain unheated/undamaged. This may allow some parts of the polymer to hit very high temperatures quickly, and then cool down without heating adjacent areas to temperatures that can damage them.

While the example IMD 30 shown in FIGS. 3A-3D is configured such that there is a lap joint or half lap joint between polymer housing component 31A and metal housing component 32, other joints types may be employed. In some examples, a butt joint, tee joint, edge joint, or the like may be used.

### EXAMPLES

Various experiments were carried out to evaluate one or more aspects of the disclosure. In each of the experiments, a titanium housing component, having an outer diameter of 0.748 inches and a wall thickness of 0.010 inches, was positioned adjacent to a polyethyl ethyl ketone polymer housing component having a diameter of 0.728 inches, a length of 0.125 inches, and an outer diameter of 0.748 inches. A laser welding process was used to heat the metal housing component which in turn heated the polymer housing component. The laser was operated in a continuous mode with a power of 200 watts and a beam diameter of 287 micrometers (11.3 mils). Weld speeds were varied for three separate samples and are included in the Table below.

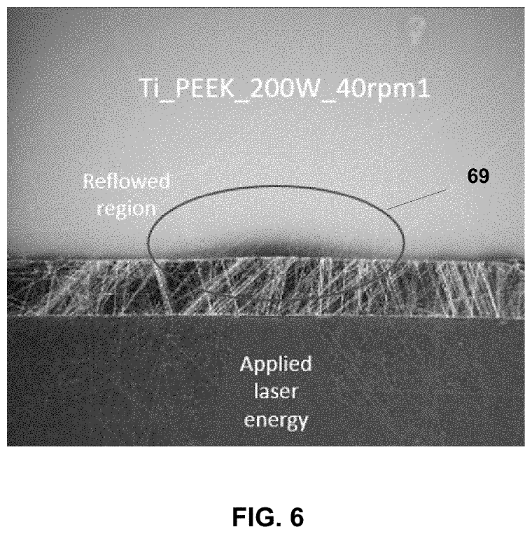

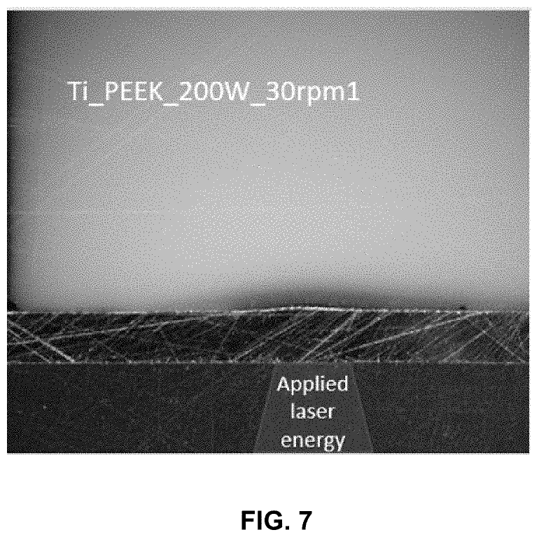

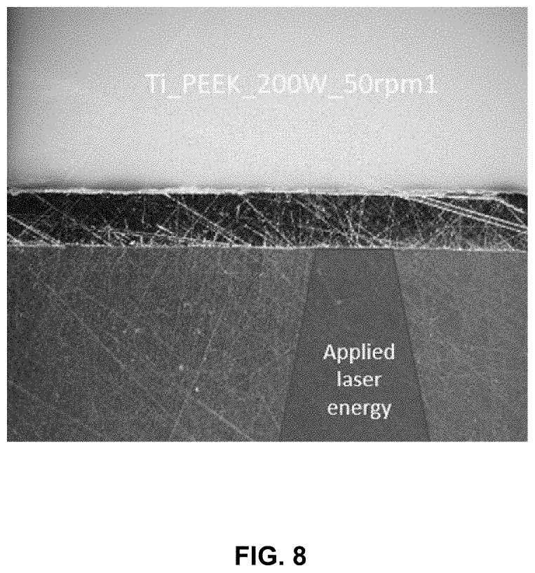

Results are summarized in the Table below. FIGS. 6-8 are micrographs showing cross-sectional views of the samples: FIG. 6 for Sample A, FIG. 7 for Sample B, and FIG. 8 for Sample C. Region 69 in FIG. 6 shows a region where material has reflowed. In each of FIGS. 6-8, the polymer component is on the “top” side and the metal component is on the “bottom” side. The region of the applied laser energy is shown in FIGS. 6-8. As illustrated in FIGS. 6-8, the two components were intimate contact to facilitate bonding during the laser welding process.

| | Rotary Weld | Weld Speed | | Hermeticity |
|---|---|---|---|---|
| Sample | Speed (rpm) | (ipm) | Observation | Check |
| A | 40 | 94.0 | THC melted on small | Hermetic |
| | | | section of edge | |
| B | 30 | 70.5 | THC melted on edge | Hermetic |
| C | 50 | 117.5 | No visible melt on | Leaked |
| | | | THC | |

One skilled in the art will appreciate that the present disclosure may be practiced with examples other than those disclosed herein. The disclosed examples are presented for purposes of illustration and not limitation, and the present disclosure is intended to be limited only by the claims, including insubstantial changes therefrom.

Various examples have been described in the disclosure. These and other examples are within the scope of the following clauses and claims.

Clause 1. A method for manufacturing an implantable medical device, the method comprising: positioning a metal housing component adjacent to a polymer housing component so that there is an interface between the metal housing component and the polymer housing component; and forming a seal at the interface between the metal housing component and the polymer housing component to join the metal housing component and the polymer housing component, wherein the joined metal housing component and the polymer housing component form at least a portion of housing for the implantable medical device, wherein the housing of the implantable medical device contains electronic circuitry.

Clause 2. The method of clauses 1 or 2, wherein positioning the metal housing adjacent to the polymer housing comprises contacting a surface of the metal housing component with a surface of the polymer housing component at the interface.

Clause 3. The method of any one of clause 1-3, wherein forming the seal at the interface between the metal housing component and the polymer housing component comprises: delivering energy to the metal housing component such that the metal housing component causes a portion of the polymer housing component to melt, wherein the melting of the portion of the polymer housing component increases contact between the metal housing component and the polymer housing component at the interface, and wherein the seal is formed between the metal housing component and the polymer housing component at the interface upon cooling of the melted portion of the polymer housing component.

Clause 4. The method of clause 3, wherein delivery in the energy to the metal housing component comprises delivering laser beam energy to the metal housing component.

Clause 5. The method of clause 4, wherein delivering the laser beam energy comprises delivering pulsed laser beam energy.

Clause 6. The method of clause 4, wherein delivering the laser beam energy comprises delivering continuous wave laser beam energy.

Clause 7. The method of clause 4, wherein the polymer housing component and the metal housing component are stationary during the delivery of the laser beam energy.

Clause 8. The method of any one of clauses 1-7, wherein the seal is a hermetic seal.

Clause 9. The method of any one of clauses 1-8, wherein positioning the metal housing component adjacent to the polymer housing component comprises forming a press fit of between the metal housing component and polymer housing component.

Clause 10. The method of any one of clauses 1-9, wherein the metal housing component comprises at least one of stainless steel, titanium, platinum, or iridium.

Clause 11. The method of any one of clauses 1-10, wherein the polymer housing component comprises polyether ether ketone.

Clause 12. The method of any one of clauses 1-11, wherein the polymer housing component comprises a liquid crystalline polymer.

Clause 13. The method of any one of clauses 1-12, wherein the polymer housing component comprises a polymer having a glass transition temperature (Tg) of less than about 150 degrees Celsius.

Clause 14. The method of any one of clauses 1-13, wherein the electronic circuitry is contained within the housing upon positioning the polymer housing component adjacent to the metal housing component.

Clause 15. The method of any one of clauses 1-14, wherein the polymer housing component includes an electrode on an outer surface of the housing.

Clause 16. The method of any one of clauses 1-15, wherein the implantable medical device comprises a cardiac monitor configured to sense and record cardiac electrogram signals.

Clause 17. An implantable medical device comprising: electronic circuitry; and a housing, wherein the processing circuitry is contained within the housing, wherein the housing includes a metal housing component and a polymer housing component sealed to each other along an interface.

Clause 18. The implantable medical device of clause 17, wherein the metal housing component comprises at least one of stainless steel, titanium, platinum, or iridium.

Clause 19. The implantable medical device of clauses 17 or 18, wherein the polymer housing component comprises polyether ether ketone.

Clause 20. The implantable medical device any one of clauses 17-19, wherein the polymer housing component comprises a liquid crystalline polymer.

Clause 21. The implantable medical device of any one of clauses 17-20, wherein the housing further comprises an electrode forming an outer surface of the implantable medical device.

Clause 22. The implantable medical device of clause 21, wherein the electrode is located on the polymer housing component, wherein the polymer housing component electrically isolates the electrode from the metal housing component.

Clause 23. The implantable medical device of any one of clauses 17-22, wherein the implantable medical device comprises a cardiac monitor configured to sense and record cardiac electrogram signals.

Clause 24. The implantable medical device of any one of clauses 17-23, wherein the seal comprises a hermetic seal.

What is claimed is:

1. A method for manufacturing an implantable medical device, the method comprising:
positioning a metal housing component adjacent to a polymer housing component so that there is an interface between the metal housing component and the polymer housing component; and
forming a seal at the interface between the metal housing component and the polymer housing component to join the metal housing component and the polymer housing component, wherein the joined metal housing component and the polymer housing component form at least a portion of housing for the implantable medical device, wherein the housing of the implantable medical device contains electronic circuitry.

2. The method of claim 1, wherein positioning the metal housing adjacent to the polymer housing comprises contacting a surface of the metal housing component with a surface of the polymer housing component at the interface.

3. The method of claim 1, wherein forming the seal at the interface between the metal housing component and the polymer housing component comprises:
delivering energy to the metal housing component such that the metal housing component causes a portion of the polymer housing component to melt, wherein the melting of the portion of the polymer housing component increases contact between the metal housing component and the polymer housing component at the interface, and wherein the seal is formed between the metal housing component and the polymer housing component at the interface upon cooling of the melted portion of the polymer housing component.

4. The method of claim 3, wherein delivery in the energy to the metal housing component comprises delivering laser beam energy to the metal housing component.

5. The method of claim 4, wherein delivering the laser beam energy comprises delivering pulsed laser beam energy.

6. The method of claim 4, wherein delivering the laser beam energy comprises delivering continuous wave laser beam energy.

7. The method of claim 4, wherein the polymer housing component and the metal housing component are stationary during the delivery of the laser beam energy.

8. The method of claim 1, wherein the seal is a hermetic seal.

9. The method of claim 1, wherein positioning the metal housing component adjacent to the polymer housing component comprises forming a press fit of between the metal housing component and polymer housing component.

10. The method of claim 1, wherein the metal housing component comprises at least one of stainless steel, titanium, platinum, or iridium.

11. The method of claim 1, wherein the polymer housing component comprises polyether ether ketone.

12. The method of claim 1, wherein the polymer housing component comprises a liquid crystalline polymer.

13. The method of claim 1, wherein the polymer housing component comprises a polymer having a glass transition temperature (Tg) of less than about 150 degrees Celsius.

14. The method of claim 1, wherein the electronic circuitry is contained within the housing upon positioning the polymer housing component adjacent to the metal housing component.

15. The method of claim 1, wherein the polymer housing component includes an electrode on an outer surface of the housing.

16. The method of claim 1, wherein the implantable medical device comprises a cardiac monitor configured to sense and record cardiac electrogram signals.

17. An implantable medical device comprising:
electronic circuitry; and
a housing, wherein the processing circuitry is contained within the housing, wherein the housing includes a metal housing component and a polymer housing component sealed to each other along an interface.

18. The implantable medical device of claim 17, wherein the metal housing component comprises at least one of stainless steel, titanium, platinum, or iridium.

19. The implantable medical device of claim 17, wherein the polymer housing component comprises polyether ether ketone.

20. The implantable medical device of claim 17, wherein the polymer housing component comprises a liquid crystalline polymer.
